(19) United States
(12) Patent Application Publication
(10) Pub. No.: US 2004/0063994 A1
(43) Pub. Date: Apr. 1, 2004

(54) Process for the preparation of cyclic amino acids

(76) Inventors: Paolo Rossi, Garlasco (IT); Emilia Vecchio, Cernusco Sul Naviglio (IT)

Correspondence Address: Walter H Schneider, 21350 Beechwood Rd, Circleville, OH, 43113, US

(21) Appl. No.: 10/466,621

(22) PCT Filed: Mar. 13, 2002
(86) PCT No.: PCT/EP02/02765

(30) Foreign Application Priority Data
Mar. 16, 2001 (IT) MI2001A000556
Jan. 1, 2002 (IT) MI2002A000103

(51) Int. Cl. C07C 327/24; C07C 229/10

(52) U.S. Cl. 560/125; 562/507; 564/191

(57) ABSTRACT

Amino Acids of formula (1) having high purity, free from the corresponding lactams and chloride anions obtained by reduction of oxyaminoacids of formula (II)

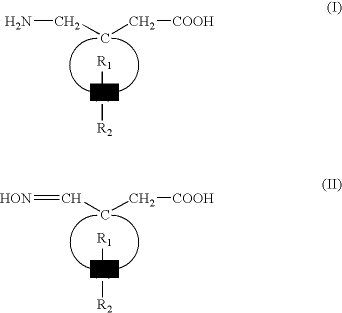

The present invention relates to a process for the preparation of highly pure cyclic amino acids, or the derivatives thereof, of formula (I)

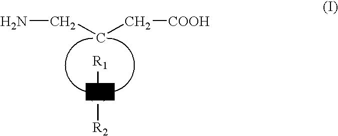

wherein:

R₁ and R₂, which can be the same or different, are hydrogen, lower alkyl or aryl;

R₃ is OH, NH₂ or lower alkoxy;

n is an integer of 3 to 11.

Said compounds are used in therapy in the neurological field; the most used among them being gabapentin (formula I, wherein R₁=R₂=hydrogen, R₃=OH and n=5).

In the compounds of formula (I), alkyl is preferably C₁-C₄ alkyl; aryl is preferably a phenyl, optionally substituted with one to three halogen atoms, C₁-C₃ alkli, nitro, cyano, C₁-C₃ alkoxy, amino group; alkoxy is preferably C₁-C₄ alkoxy and n is preferably an integer of 4 to 7. Preferred compounds of formula (I) are those in which the groups R₁ and R₂ are hydrogen.

### PRIOR ART

U.S. Pat. No. 6,103,932 surrrarizes some known synthetic routes for the preparation of cyclic amino acids. All of these routes start from cyclohexanone and involve the use of sodium cyanide or triphenylphosphine derivatives, followed by hydrolysis and decarboxylation of the nitrites. They involve a number of steps, give rather low yields and require difficult, costly purifications. The procedures currently used for preparing gabapentin are summarized hereinbelow.

Method 1 (U.S. Pat. No. 4,024,175).

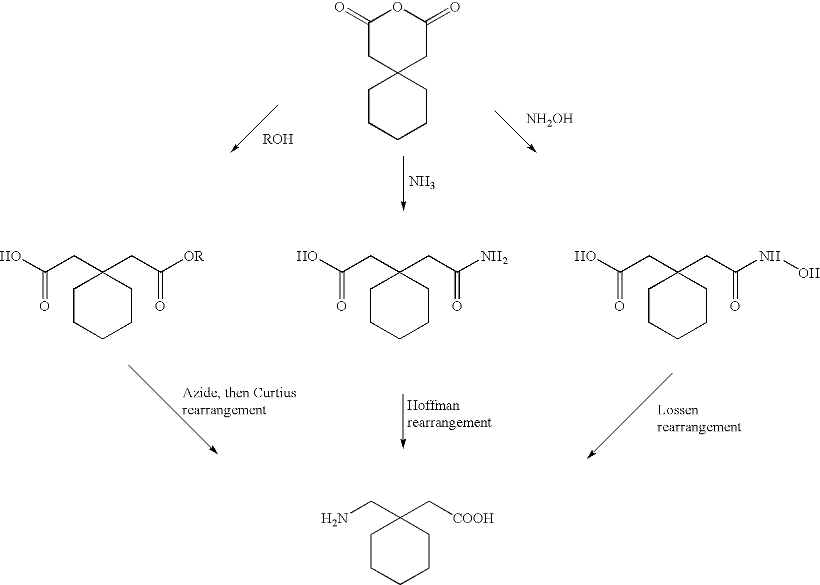

Gabapentin is then transformed into a pharmacologically compatible salt by reaction with either acids or bases. The starting cyclic anhydride can be prepared through various steps starting from cyclohexanone (JCS 115, 686, 1919).

The method according to U.S. Pat. No. 4,024,175 is industrially expensive in that it involves a remarkable number of steps as well as special safety measures for handling azides and isocyanates. For this reason, the

Method 2 (Helv. Chim. Acta 74,2,1991, 309) has been suggested:

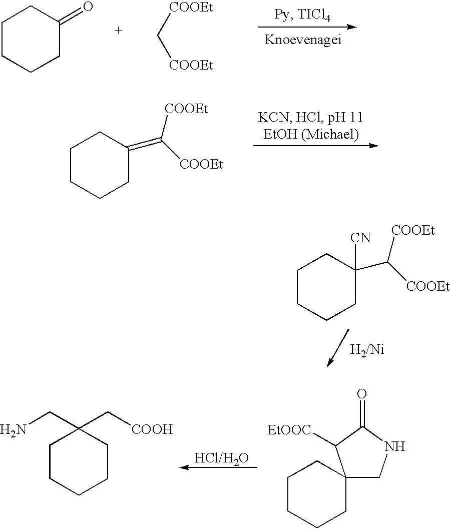

However, this process is not economically advantageous, since the Knoevenagel reaction requires two TiCl₄ equivalents and four pyridine equivalents, whereas yields from the Michael reaction are only acceptable when using 1.5 KCN equivalents. The process is therefore expensive for both safety reasons and the large amount of solvent required. These problems have been solved by

Method 3 (Helv. Chim. Acta, loc. cit.):

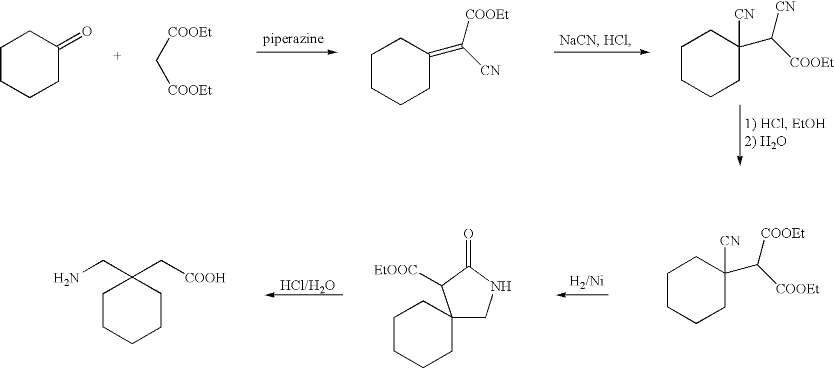

During the hydrolysis reaction of the cyano group, the substrate is not isolated as it undergoes decomposition:

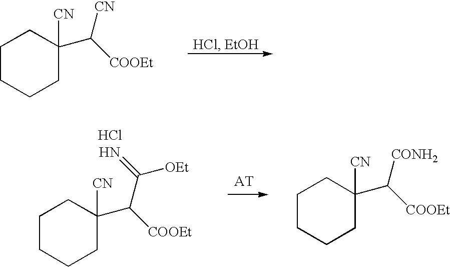

This method provides gabapentin as the hydrochloride, which has to be treated with a ion exchange resin. To avoid this operation, the following route may be followed:

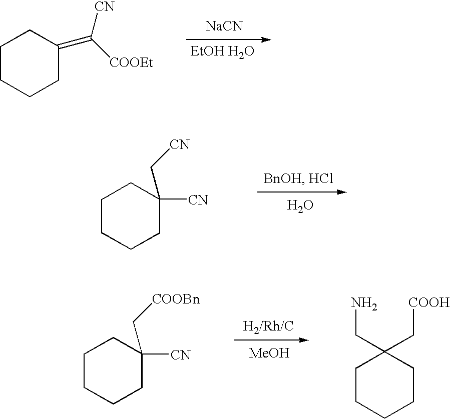

(wherein Bn=benzyl)

The last step, however, provides only 27% yield, and an excess of benzyl alcohol, which is rather expensive, is necessary.

### DISCLOSURE OF THE INVENTION

According to the present invention, the compounds of formula (I) are prepared starting from aldehydes of formula (II)

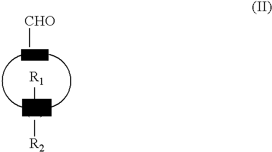

(where R₁, R₂ and n are as defined above)

which are converted into the corresponding enamines of formula (III)

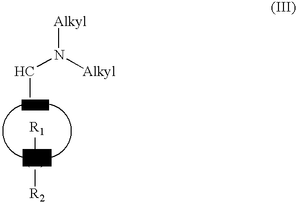

wherein R₁, R₂ and n are as defined above;

said enamines are then reacted with α-chloro- or α-bromo- acetic acid esters or amides to give, after hydrolysis of the enamino group, aldehydes of formula (IV)

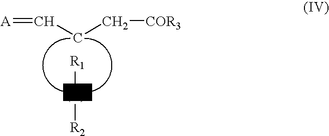

wherein

A=OR;

R₃ OR₄ (R₄ being C₁-C₄ alkyl) or NH₂;

R₁, R₂ and n are as defined above.

According to an embodiment of the invention, the aldehydes can be transformed into the corresponding compounds with A=NCH₂Ar by reaction with aralkylamines, or into the corresponding compounds with A=(OR₄)₂ by reaction with C₁-C₄ aliphatic alcohols.

According a preferred embodiment of the invention, however, the aldehydes are reacted with hydroxylamine to give the corresponding oximes of formula (IV) with A=NOH which can be reduced to the compounds of formula (I) wherein R₃=C₁-C₄ alkoxy. Finally, the ester group is hydrolyzed to yield amino acids of formula (I), wherein R₄=OH.

This embodiment of the invention will be now illustrated in more detail with reference to the preparation of 1-(aminomethyl)-cyclohexaneacetic acid (Gabapentin, formula I wherein R₁=R₂=H, n=5, R₃=OH).

Starting hexahydrobenzaldehyde is dissolved in an aromatic solvent, such as benzene or preferably toluene, then added with a secondary amine, preferably diisobutylamine, and refluxed removing the formed water, to obtain the corresponding enamine. When water no longer forms, methyl, ethyl or propyl α-bromoacetate or α-chloroacetate and an aprotic polar solvent, such as dimethylformamide, dimethylacetamide, acetonitrile (preferably the latter) are added to the mixture, which is heated for a further 40 hours. The enamine moiety is then hydrolyzed by adding to the hot solution a weak acid, such as aqueous acetic acid or aqueous propionic acid, preferably aqueous acetic acid. The mixture is then cooled and diluted with water. The organic phase is washed with diluted hydrochloric acid or with diluted sulfuric acid, then with sodium carbonate. The solvent is evaporated off and the residue is fractionated under vacuum, to obtain the 1-(formyl)-cyclohexaneacetic acid ester as a substantially pure colorless distillate.

The corresponding oxime is prepared by adding the formyl ester to an aqueous suspension of hydroxylamine hydrochloride and sodium or potassium carbonate in stoichiometrically equivalent amounts, preferably with an about 0.1 molar excess of sodium or potassium carbonate, the molar ratio of the amount of bydroxylamine formed from the hydrochloride by action of the carbonate to the formyl ester ranging from 1:1 to 1.5:1, preferably 1.1:1.

The mixture is stirred at 30-50° C., preferably at 40° C., until gaschromatographic analysis shows disappearance of the formyl ester and formation of the corresponding oxime. After completion of the reaction, the mixture is extracted with ethyl acetate, the extract is washed with water and the solvent is evaporated off to an oily residue.

The resulting alkyl 1-(oxyiminomethyl)-cyclohexaneacetate is then transformed into the corresponding alkyl 1-(aminomethyl)-cyclohexaneacetate by reduction, for example by catalytic hydrogenation. For this purpose, the oxime is dissolved in a dry alcoholic solvent, preferably a tertiary alcohol, more preferably tert-butyl alcohol. The concentration of the oxime in solvent can range from 5 to 50%, preferably from 10 to 30% (w/v). The alcoholic solution is saturated while cold with gaseous ammonia, added with a hydrogenation catalyst, such as nickel Raney, 5 or 10% palladium on charcoal or rhodium on allumina. 5 To 10% rhodium on allumina is preferably used. The ratio of catalyst to oxime solution may range from 0.2 to 20%, preferably from 0.5 to 10% (w/v). Hydrogenation is carried out under hydrogen pressure of 3 to 50 atm, preferably 5 to 30 atm. Temperature may range from 20 to 60° C., preferably from 30 to 50° C.

When hydrogen absorption ceases, the catalyst is filtered off and 15-30% sodium or potassium hydroxide is added, so that the NaOH/aminoester (or KOH/aminoester) molar ratio is 0.9 to 2 NaOH or KOH mols per mol of ester, preferably 1.0 to 1.3 mols per mol, heating to ebullition to hydrolyze the ester group. Conc. HCl or conc. H₂SO₄ is added, preferably conc. HCl, in equimolar amount to the added NaOH or KOH. The formed sodium chloride is filtered from the hot mixture. Water is distilled off under reduced pressure until incipient crystallization, the first crystals are filtered from the hot solution and discarded, and the filtrate is cooled. Alternatively, after the neutralization with acid, the mixture is evaporated to small volume, treated with a low-boiling alcohol (methanol or ethanol), the crystals of the inorganic salt are filtered off, the mixture is concentrated to small volume again and taken up with alcohol and active charcoal. The inorganic crystals are filtered off, the mixture is concentrated and left to stand to crystallize Gabapentin hydrochloride. Gabapentin may be obtained from the latter by treatment with a ion exchange resin, e.g. as disclosed in U.S. Pat. No. 6,054,482.

With the above described methods for the preparation of Gabapentin, also other compounds of formula (I) and (II) may be obtained, starting from the suitable intermediates. For example, in the case of compounds of formula (I) wherein n=7 and R₁=R₂=H, the starting aldehyde is formylcyclooctane, obtainable for example from cyclooctane by hydroformylation. The corresponding enamine is reacted with α-methyl bromoacetate, to yield methyl 1-(formyl)-cyclooctaneacetate, from which 1-(aminom ethyl)-cyclooctaneacetic acid is prepared, analogously to the procedure described above.

Other aldehydes suitable as starting materials for preparation of compounds of formula (I) include, for example, 1-formyl-4-methylcyclohexane or 1-formyl 3-methylcyclohexane, obtained from m-toluic or p-toluic acids via hydrogenation of the aromatic ring, followed by chlorination of the carboxyl to acid chloride and reduction according to Rosenmund. The procedure as described above is followed in this case as well. The preferred α-haloacid is α-ethyl bromoacetate. In this case, compounds of formula (I) are obtained in which n is 5 and one of the groups R₁ or R₂, at the specified positions, is methyl whereas the other is hydrogen.

According to the above illustrated processes, Gabapentin (see U.S. Pat. No. 6,054,482) with very low content of Cl⁻ and of the corresponding lactam can be obtained. However, the final steps are industrially complex and expensive, since remarkable amounts of water have to be evaporated and passages on resins are required to minimize the amount of chloride ions present.

A further characteristic shared by the known processes used for the preparation of amino acids (I) is the concomitant formation of the corresponding lactams, in varying proportions.

According to a particularly preferred embodiment of the present invention, cyclic amino acids of formula (I), particularly Gabapentin, may be prepared, which has high purity and is completely free from both anions, in particular chloride ions, and the corresponding lactams, by subjecting to either reduction or catalytic hydrogenation compounds of formula (V)

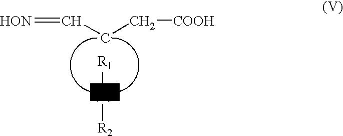

wherein R₁, R₂ and n have the meanings defined above.

The purity of the resulting amino acids (I) can be further increased by recrystallization from usual solvents, such as methanol, ethanol, isopropanol or mixtures thereof, in the absence of any acid.

Compounds (V) can in turn be obtained from the corresponding aldehyde-acids of formula (IV) wherein A=OR and R₃=OH (whereas R₁, R₂ and n have the meanings defined above) by reaction with hydroxylamine; or, alternatively, from oxyimino-esters of formula (IV) wherein A=NOH and R₃=lower alkoxy (whereas R₁, R₂ and n have the meanings defined above) by basic hydrolysis of the ester group.

In both cases, the oxyimino-acids (V) may be isolated as such, free from inorganic anions, from the solutions of the corresponding alkali salts by precipitation at pH 4-5.

This preferred process of the invention will be now illustrated with reference to the preparation of Gabapentin. The procedure illustrated above is followed, until obtaining the alkyl 1-(oxyiminomethyl)-cyclohexaneacetate; the subsequent hydrolysis with sodium or potassium hydroxide yields 1-(oxyiminomethyl)-cyclohexaneacetic acid (formula IV, with A=NOH, R₃OH, R₁=R₂=H, n=5). Said compound is preferably obtained, however, according to the invention starting from the corresponding aldehydo-acid (formula IV, with A=CHO, R₃=OH, R₁=R₂=H, n=5).

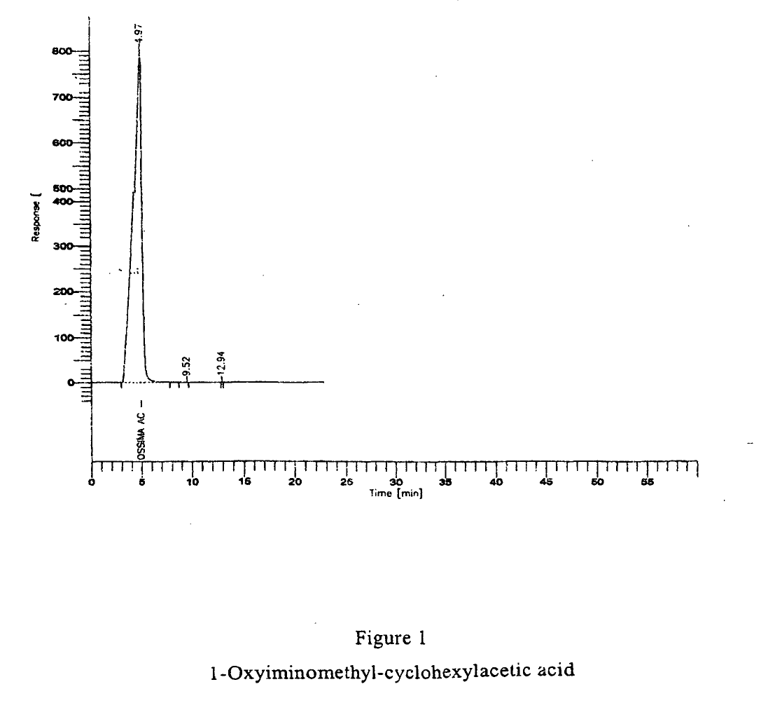

For this purpose, alkyl 1-(formyl)-cyclohexaneacetate is reacted with a sodium or potassium hydroxide aqueous solution, preferably sodium hydroxide, in about equimolar amounts, preferably in a 10% molar excess, stirring at 10-50° C., preferably at 20-30° C., for some hours. After completion of the hydrolysis, the mixture is acidified with concentrated hydrochloric acid, diluted sulfuric acid or acetic acid, preferably with hydrochloric acid, to final pH 7.65, then sodium or potassium carbonate, preferably sodium carbonate, in a 0.1 molar excess to the starting formyl ester, preferably in 0.2 is molar excess, and hydroxylamine hydrochloride in 0.1 molar excess, are added. Upon completion of the reaction, the mixture is acidified with concentrated hydrochloric acid or sulfuric acid or phosphoric acid, preferably concentrated hydrochloric acid, to pH 4, stirring and then filtering the crystals, which are washed with distilled water. The resulting 1-(oxyimino)-cyclohexaneacetic acid has HPLC purity =100% (FIG. 1).

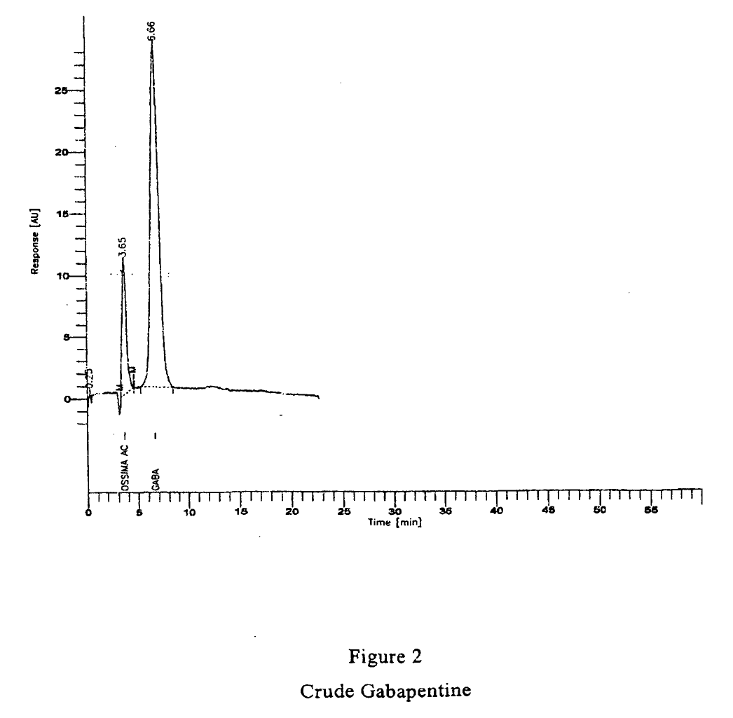

Said compound can then be transformed into the corresponding amino acid by reduction, for example by catalytic hydrogenation. For this purpose, the compound is dissolved in an alcoholic aqueous solvent, preferably a low-boiling alcohol, more preferably methanol. The concentration of the oxime in the alcoholic solvent can range from 5 to 50%, preferably from 10 to 30% (w/v). The alcoholic solution is added with a hydrogenation catalyst such as nickel Raney, 5 or 10% palladium on charcoal or, preferably, 5 or 10% rhodium on allumina. The amount of catalyst in the oxime alcoholic solution can range from 0.2 to 20%, preferably from 0.5 to 10% (w/v). Hydrogenation is carried out under hydrogen pressure of 3 to 50 atm, preferably under 5 to 30 atm. Temperature can range from 0 to 100° C., preferably from 10 to 50° C. When hydrogen absorption ceases, the catalyst is filtered off and the mixture is concentrated under vacuum at a temperature of 20 to 60° C., preferably 30 to 50° C. The residue is taken up with acetone or methyl ethyl ketone or methyl isobutyl ketone, preferably acetone, and filtered, thereby obtaining the crude amino acid with HPLC purity >98% (FIG. 2).

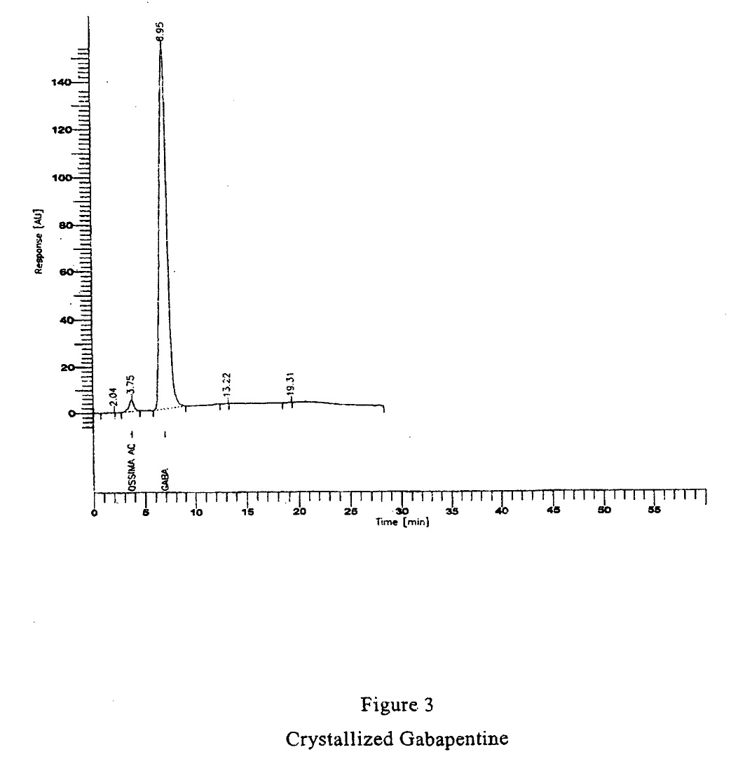

The crude is taken up with 10 volumes of methanol or ethanol or isopropanol, preferably hot methanol; the solution is decolorized with active charcoal and filtered. The filtrate is added with about 10 volumes of isopropanol and cooled at −5 to +10° C., preferably between −5 and 0° C., keeping said temperature for 3 hours. The mixture is then filtered and washed with fresh isopropanol, thereby obtaining Gabapentin with HPLC purity >99.8% (FIG. 3), total absence of inorganic anions and lactam and yields higher than 70% compared with starting oxyiminoacid.

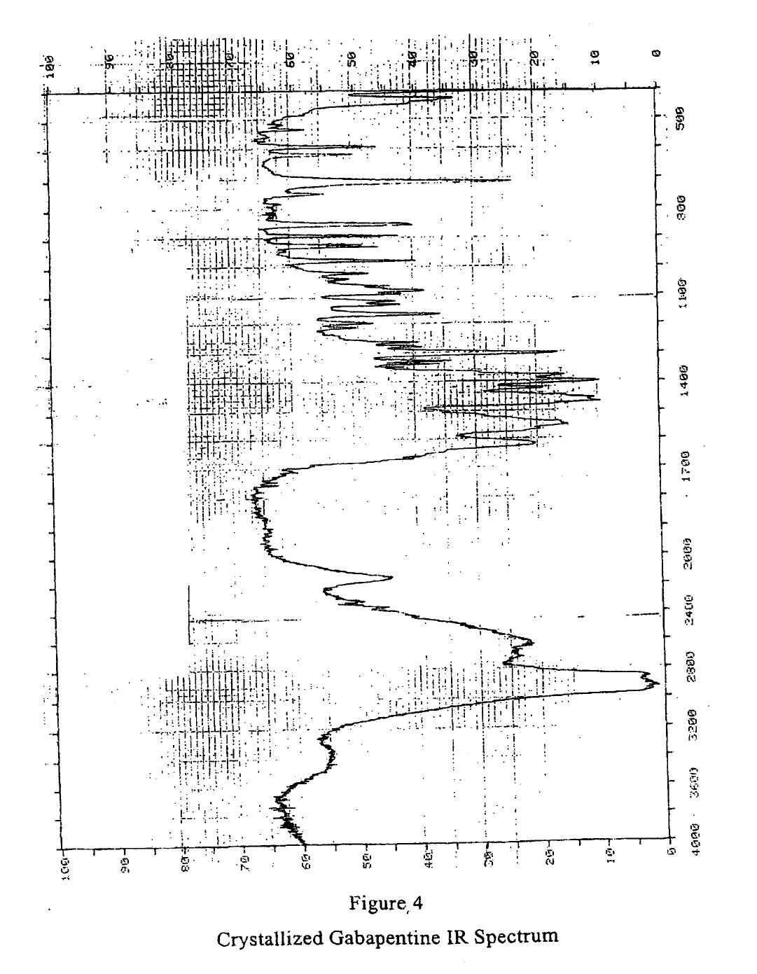

The IR spectrum (FIG. 4) shows peaks at 709, 748, 854, 929, 977, 1165, 1300, 1421, 1466, 1548 and 1615 cm⁻¹.

The invention is described in greater detail in the following examples.

### EXAMPLE 1

Ethyl 1-(formyl)-cyclohexaneacetate.

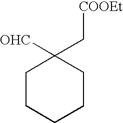

92.4 g of hexahydrobenzaldehyde (0.825 mols), 106.5 g of diisobutylamine (0.825 mols) and 250 ml of toluene are refluxed, continuously removing the formed water by azeotropical distillation. Upon completion of the reaction (about 12 hours), the mixture is cooled to 80-90° C. and added first with 207.1 g of ethyl bromoacetate, subsequently with 200 ml of acetonitrile, refluxing for 40 h. After this time, the hot solution is added with 198 ml of aqueous acetic acid (33% volume of CH₃COOH). After hot hydrolysis for 3 h the mixture is cooled and the phases are separated. The organic phase is added with a solution of 250 g of aqueous HCl formed by 50 g of conc. HCl and 200 g of water. The hydrochloric aqueous phase is separated and the organic phase is washed with water to neutrality, then evaporated under vacuum and the residue is fractionated under vacuum, to obtain, as the main fraction, 113.8 g (60%) of an oil boiling at 120° C. under 1.5 mm Hg, which consists of ethyl 1-(formyl)-cyclohexaneacetate.

H-NMR: δ: 1.2 (3H triplet) δ: 1.3-1.55 (8H multiplet) δ: 1.8 (2H multiplet) 8:2.5 (2H singlet) δ: 9.7 (1H singlet).

### EXAMPLE 2

Ethyl 1-(oxyiw-inomethyl)-cyclohexaneacetate.

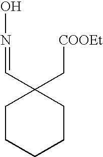

40 g (0.22 mols) of the compound obtained in example 1, 16.8 g (0.242 mols) of hydroxylamine hydrochloride and 12.8 g of sodium carbonate dissolved in 100 ml of water are placed in a flask equipped with magnetic stirrer. Stirring is continued for, two hours at room temperature, then the mixture is extracted with 2×100 ml of ethyl acetate. The organic phase is washed with water and evaporated to dryness, to obtain 42 g of a colorless oil which shows by GLC 95% purity.

### EXAMPLE 3

Ethyl 1-(oxyiminomethyl)-cyclohexaneacetate

14 g (0.2 mols) of hydroxylamine hydrochloride are dissolved under stirring in 30 ml of distilled water and 30 ml of methanol. 11 g (0.1 mols) of sodium carbonate are added thereto in portions. The mixture is stirred for about 30 minutes, then added with 20 g (0.1 mols) of ethyl 1-(formyl)-cyclohexaneacetate. After stirring overnight, 40 ml of distilled water are added and the mixture is extracted with 2×70 ml of ethyl acetate. The combined extracts are washed with 30 ml of distilled water, then the solvent is evaporated off under vacuum to constant weight, to obtain 19.8 g (0.93 mols) of title product.

### EXAMPLE 4

1-(Aminomethyl)-cyclohexaneacetic Acid

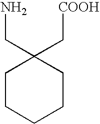

40 g of ethyl 1-(oxyiminomethyl)-cyclohexaneacetate (0.2 mols) are dissolved in 200 ml of ethyl alcohol. The cold solution is added with 17 g of gaseous ammonia and 10 g of 5% rhodium on allumina. The mixture is placed in a glass autoclave and hydrogenated at 60° C. under hydrogen pressure of 9 atm. When the absorption ceases, the autoclave is cooled, washed with nitrogen and the catalyst is filtered off. The alcoholic solution is added with a solution of 0.2 mols of NaOH in 18 ml of water and refluxed for 4 hours. After that, the mixture is cooled, added with 0.2 mols of HCl in 18 ml of water, methanol, and sodium chloride is filtered off. The filtrate is evaporated to small volume, dissolved again in ethanol, treated with active charcoal and filtered. The solution is saturated with gaseous HCl, concentrated and cooled to crystallize 30.5 g of 1-(aminomethyl)-cyclohexaneacetic acid hydrochloride, m.p. 120-124° C.

H-NMR: δ: 1.3-1.5 (10H multiplet) δ: 2,4 (2H singlet) δ: 2.9 (2H quartet) δ: 8 (2H singlet)

C-NMR 21,3-25.4 -33.7 (cyclohexane CH₂); 35,1 (C quaternary); 39,6 (C secondary); 47,0 (C secondary); 176,4 (C carbonyl).

### EXAMPLE 5

1-(Oxyiminometyl)-cyclohexaneacetic Acid

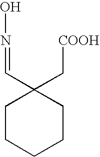

The compound obtained in example 3 is added with 50 ml of distilled water and cooled on an ice-bath to 10° C. 13 g (0.1 mols) 30% sodium hydroxide solution are slowly added in 15 minutes. After completion of the addition, the mixture is kept at 10° C., monitoring the progress of the hydrolysis by HPLC.

The reaction leads to simultaneous hydrolysis of about 25% of oxime to aldehyde. 3.5 g (0.05 mols) of sodium carbonate and 7 g (0.1 mols) of hydroxylamine hydrochloride are added and the mixture is stirred for about one hour. pH is adjusted to 5 with 30% HCl and the mixture is extracted with 2×60 ml of isobutanol. The combined organic phases are washed with 30 ml of distilled water to obtain, after evaporation, 15.6 g (0.845 mols) of the title compound in a 91% yield. No traces of chlorides can be detected.

### EXAMPLE 6

1-(Oxyiminomethyl)-cyclohexaneacetic Acid

A mixture of 40 g (0.2 mols) of ethyl 1-(formyl)-cyclohexaneacetate prepared as in example 1, 200 ml of distilled water and 35 g of 40% sodium hydroxide solution (0.26 mols) stirred at room temperature. After completion of the hydrolysis, the mixture is acidified with 30% HCl to final pH 7.65. 16 g of sodium carbonate and 20.7 g (0.29 mols) of hydroxylamine hydrochloride are added to the mixture, which is stirred for 30 minutes, then acidified to to pH 4 with 30% HCl and left under stirring overnight. After that, the mixture is filtered by suction and washed with distilled water, to obtain 32 g (0.17 mols) of title product in a 90% yield and 100% HPLC purity.

Total absence of chlorides.

### EXAMPLE 7

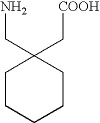

1-(Aminomethyl)-cyclohexaneacetic Acid

10 g (0.054 mols) of 1-(oxyminomethyl)-cyclohexaneacetic acid prepared as in example 5, 8 ml of distilled water and 75 ml of isopropanol are stirred to complete dissolution. The solution is placed in autoclave, added with 1 g of 5% Rh/Al₂O₃ and hydrogenated at a temperature of 20° C. and under hydrogen pressure of 9 atm. When the absorption of hydrogen ceases, the catalyst is filtered off and the solution is concentrated under vacuum at temperature below 40° C.

The residue is taken up into acetone and filtered, to obtain 8.2 g (0.047 mols) of gabapentin of HPLC purity higher than 98%.

The crude is dissolved in 10 volumes of hot methanol, treated with active charcoal and Celite and filtered while hot. The filtrate is added with 10 volumes of isopropanol and cooled to 0° C. for 3 hours. The mixture is filtered and the filtrate is washed with fresh isopropanol, to obtain 6.1 g of gabapentin with m.p. 164, HPLC purity >99.8 and total absence of chlorides and lactam.

H-NMR: δ: 1.3-1.5 (10H multiplet) δ: 2.4 (2H singlet) δ: 2.9 (2H quartet) δ: 8 (2H singlet).

A second crop of the product can be obtained by concentrating the mother liquors.

1. A process for the preparation of highly pure cyclic amino acids, or the derivatives thereof, of formula (I) 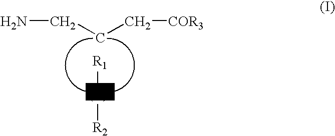
wherein:
R₁ and R₂, which can be the same or different, are hydrogen, lower alkyl or aryl;
R₃ is OH, NH₂ or lower alkoxy;
n is an integer of 3 to 11,
which process comprises reducing compounds of formula (IV) 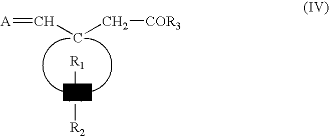
wherein
A=OR;
R₃=OR₄ (R₄ being C₁-C₄ alkyl) or NH₂;
R₁, R₂ and n are as defined in formula (I),
then hydrolyzing the OR₃ group to OH group.

2. A process for the preparation of cyclic amino acids, or the derivatives thereof, of formula (I) 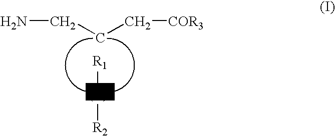
wherein
R₁ and R₂, which can be the same or different, are hydrogen, lower alkyl or aryl;
R₃ is OH, NH₂ or lower alkoxy;
n is an integer of 3 to 11,
which compounds are free from the corresponding lactams and mineral acid anions,
which process comprises reducing compounds of formula (V) 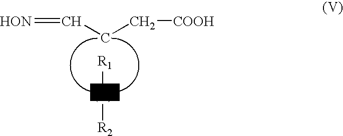
wherein R₁, R₂ and n have the meanings defined above, in the absence of mineral acids, and the resulting amino acids are purified by simple crystallization from conventional solvents, in the absence of any acid.

3. A process as claimed in claims 1-2, wherein the reduction is carried out by catalytic hydrogenation.

4. A process as claimed in claim 3, wherein the catalyst is selected from the group consisting of nickel Raney, palladium on charcoal and rhodium on allumina.

5. A process as claimed in claim 4, wherein the catalyst is 5-10% rhodium on allumina.

6. A process as claimed in the above claims, wherein R₁=R₂=H and n ranges from 4 to 7.

7. A process as claimed in the above claims for the preparation of 1-(aminomethyl)-cyclohexaneacetic acid, 1-(aminomethyl)-cyclooctaneacetic acid, the C₁-C₄ alkyl esters and the amides thereof.

8. A process as claimed in any one of the above claims, wherein the compound subjected to reduction is 1-(oxyiminomethyl)-cyclohexaneacetic acid

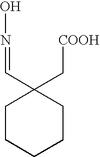

9. 1-(Aminomethyl)-cyclohexaneacetic acid free from the corresponding lactam and from inorganic acid ions.

10. A novel compound selected from the group consisting of:
1-(formyl)-cyclohexaneacetic acid;
ethyl 1-(formyl)-cyclohexaneacetate;
1-(oximinomethyl)-cyclohexaneacetic acid;
ethyl 1-(formyl)-cyclohexaneacetate.
